(19) United States
(12) Patent Application Publication
(10) Pub. No.: US 2025/0161101 A1
(43) Pub. Date: May 22, 2025

(54) Bite Savers

(71) Applicant: Michael A. Gaskill, Allison Park, PA (US)

(72) Inventor: Michael A. Gaskill, Allison Park, PA (US)

(21) Appl. No.: 18/517,495

(22) Filed: Nov. 22, 2023

(51) Int. Cl. A61F 5/56

(52) U.S. Cl. CPC A61F 5/566; A61F 2005/563

(57) ABSTRACT

BiteSavers are a pair of separate anti-bruxism mouthpieces. BiteSavers are made to function as a pair, a design trait I have not seen in any other mouthpiece. My design is more comfortable and effective than other mouthpieces I have tried because they stay in position better, are lightweight, the buffers are effective and possess a more natural jaw angle, and, because they are slim, they do not interfere as much with speech, so the user has the opportunity to speak with the mouthpieces still in their mouth.

### FIELD

The present application relates to a pair of anti-bruxism mouthpieces that function as a unit.

### BACKGROUND

Many people suffer from bruxism and the accompanying headaches, neck soreness, dental misalignment, and jaw discomfort. There are several anti-bruxism mouthpieces on the market, but BiteSavers differs from the others because of its combination of design and comfort. Many mouthpieces on the market provide a mouthful of plastic, some pop out of the mouth during sleep, some are uncomfortable, and still others don't do enough. This got me thinking about a design that would be effective, safe, comfortable, and remain in my mouth.

### SUMMARY

BiteSavers are comprised of a pair of T-shaped mouthpieces that work together-one on one side of the mouth and one on the other. They are comprised of a Bite Surface that fits between the user's teeth and an attached Retainer to ensure the device remains in position. The Retainer sits between the user's cheek and gum while the attached Bite Surface acts as a buffer to the user's teeth clenching pressure.

I will explain these devices in the following descriptions and drawings.

### BRIEF DESCRIPTION OF THE DRAWINGS

In the following detailed description, I will explain in greater detail the examples depicted in the drawings, on page 4, in which:

FIG. 1 is the Front View

FIG. 2 is the Side View

FIG. 3 is the Rear View

The dimensions of each component are provided on page 5.

### DETAILED DESCRIPTION

BiteSavers are a pair of small mouthpieces—a right and left—that work together, designed to reduce the effects of bruxism—effects such as dental misalignment, jaw discomfort, neck soreness, and headaches.

To install each mouthpiece, the user opens their mouth and slides the BiteSaver Retainer between their cheek and gum with the Bite Surface remaining between their upper and lower teeth.

The Retainer is rectangular to help maintain position and aid in the retention of the mouthpieces. They are also slim and have curved corners for comfort during installation and continuous wear.

The design is also helpful for some wearers who breathe through their mouths because of sinus congestion as it allows for a more accessible oral airway; something unsafe while using some other anti-bruxism products.

BiteSavers can be used by almost anybody as they are designed to conform to the shape of a human mouth and made of a flexible plastic or rubber material.

Some users might find it possible to speak without removing them from their mouth. This can be useful prior to sleep and upon awakening.

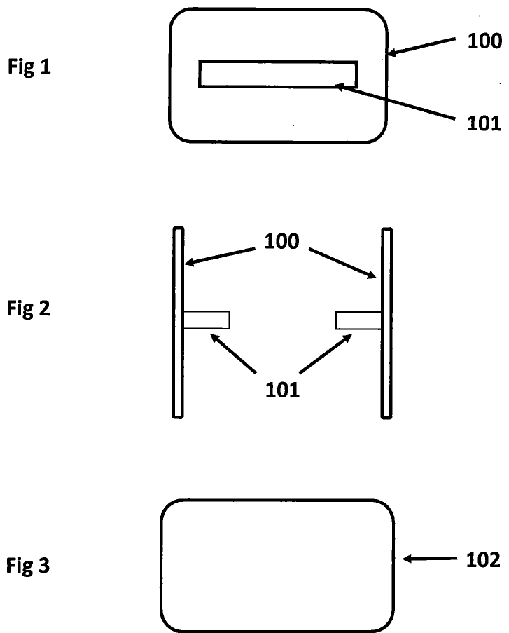

On page 4, in FIG. 1, the front view of BiteSavers is depicted with the Retainer 100 and the Bite Surface 101.

The Bite Surface 101, protrudes from the Retainer 100.

The Bite Surface is triangular to conform to the user's natural angle of jaw closure. As noted in the diagram, the wider part of the triangular Bite Surface is to be pointed toward the front of the user's mouth while the narrow part is pointed toward the rear.

FIG. 2 depicts a side view of the mouthpieces. This is how BiteSavers would be situated in one's mouth, with the horizontal rectangle representing each protruding Bite Surface 101 while the vertical rectangles represent the Retainers 100.

In FIG. 3, 102 depicts the rear of the Retainer. This part bears no prominent structure as it would rest against the inside of the user's cheek.

On page 5, I have provided the measurements for the BiteSavers mouthpieces-Front View, Side View, and Rear View-depicted with Imperial units.

Note: Only one design is required because after making, say, two ‘rights,’ one of the pair is flipped to be the ‘left.’

To make my prototypes, I utilized free online 3D printer software on my desktop PC and purchased a 3D printer to produce them from TPU filament.

Other material such as food grade silicone, EVA, or any similar material might work better, but TPU is what I have worked with to this point. The TPU version of BiteSavers has served me well.

1. BiteSavers are an anti-bruxism mouthpiece system comprised of two identical, but separate, mouthpieces that function as a pair; something no other mouthpiece I know of possesses.

2. Each of the two BiteSavers is comprised of perpendicular (T-shaped) pieces-one Retainer and one Bite Surface-something no other mouthpiece I know of possesses.

3. The two mouthpieces that comprise BiteSavers use what I call Retainers, which rest between the user's cheek and gum, that are designed to maintain retention and proper placement, something no other mouthpiece I know of possesses.
